(19) United States
(12) Patent Application Publication
(10) Pub. No.: US 2012/0220918 A1
(43) Pub. Date: Aug. 30, 2012

(54) DILATOR AND ASSEMBLY COMPRISING SUCH A DILATOR

(75) Inventor: Bernard Chaffringeon, Granges-Paccot (CH)

(73) Assignee: BE EASY IP, Capellen (LU)

(21) Appl. No.: 13/389,541

(22) PCT Filed: Aug. 13, 2010
(86) PCT No.: PCT/EP2010/061842

§ 371 Date: Apr. 23, 2012

Related U.S. Application Data
(60) Provisional application No. 61/272,095, filed on Aug. 14, 2009.
(60) Provisional application No. 61/272,678, filed on Oct. 20, 2009.
(60) Provisional application No. 61/258,859, filed on Nov. 6, 2009.
(60) Provisional application No. 61/282,987, filed on May 3, 2010.
(60) Provisional application No. 61/344,346, filed on Jul. 1, 2010.

(51) Int. Cl. A61F 13/26

(52) U.S. Cl. 604/11

(57) ABSTRACT

A dilator including a body having at least a proximal provided with a proximal end area by which can be introduced a tampon, and a distal section intended to be introduced into the vaginal wall of a user, the distal section being shaped so that it can switch from an initial resting state, in which it delimits a longitudinal internal passage, the cross-section of which is smaller than the cross-section of the tampon, to a deployed state, in which the cross-section of the internal passage adapts to the cross-section of the tampon, so as to allow for the longitudinal movement of the tampon, it is long enough to allow for release of the tampon directly into the vaginal cavity, without the tampon being able to make any prolonged contact during intromission thereof with the area of the muscular narrowing of the vaginal wall.

This invention relates to the field of period and/or hygienic tampons and to devices allowing to absorb natural body effluents, such as for instance menstruation flow and cervical mucus.

Period and/or hygienic tampons are made of absorbent material in the form of sheets and/or strips, which are folded and/or wrapped by techniques well known to the person skilled in the art for making tampons which are placed inside the user's vagina.

Such a device is commonly used no matter which volume of secretions is to be absorbed, and is generally configured so that it can retain a median or even high flow, so that it is suitable for most persons.

Some users take hold of the tampon directly and introduce it into the vaginal cavity, while others prefer to perform intromission of the tampon by means of an applicator.

On the occasion of an intromission without an applicator, the act of intromission may be painful for the user because, due to the relatively large cross-section of the tampon with regard to the introducing orifice on the one hand, and the generally rough surface of the tampon at the periphery on the other hand, the intromission of the tampon may cause lesions or irritations by friction of the vaginal wall, above all in the area of muscular narrowing of the latter.

Also, in addition to the unpleasant feeling of pain resulting from the occurrence of such lesions, it should be kept in mind that the consequences of such lesions or irritations may be dramatic. Indeed, the risk of contracting an infectious disease, namely a sexually transmissible infectious disease, then really becomes a concern.

On the occasion of an intromission by means of an applicator, the user takes hold of an assembly comprising, on the one hand, an applicator made in the form of a tube into which a tampon has been slipped, and on the other hand, a plunger inserted into the tube and intended to cause the ejection of the tampon from the applicator. In use, one kind of user will press the assembly against the genital organ so that the distal end of the tube remains near the introducing orifice of said genital organ. In this case, the disadvantages encountered are the same as those discussed previously during intromission without an applicator. Alternatively, a different kind of user will introduce the applicator deeper until reaching the vaginal cavity. In this case, the risk of the tampon rubbing against the vaginal wall will be minimized, however, the user will still be confronted with the unpleasant feeling of deeply introducing an object having a diameter substantially equal to that of the tampon.

It is an object of the present invention to eliminate all or part of the disadvantages mentioned before, and for this purpose it consists of a dilator comprising a body having at least a proximal section provided with a proximal end area by which a ready-to-use hygienic tampon can be introduced, and a distal section intended to be introduced into a user's vaginal wall, said distal section being shaped so that:
on the one hand, it can switch from an initial resting state, in which it delimits a longitudinal internal passage, the cross-section of which is smaller than the cross-section of the tampon, to a deployed state, in which the cross-section of the internal passage adapts to the cross-section of the tampon, so as to allow for the longitudinal movement of said tampon,
on the other hand, it is long enough to allow for release of the tampon directly into the vaginal cavity, without said tampon being able to make any prolonged contact during intromission thereof with the area of the muscular narrowing of the vaginal wall.

Thus, such a dilator allows to avoid the possibility of untimely rubbing of the tampon against the vaginal wall, namely within an area of muscular narrowing around the introducing orifice, due to the sleeve-type distal section thereof emerging directly into the vaginal cavity. Thereby, any risk of lesion or irritation is cancelled out.

Also, insertion of this dilator is made easy and atraumatic due to the fact that the distal section has a passage the cross-section of which in the initial resting state is minimal in comparison with the diameter of a tampon. Consequently, the distal section may be slipped through the introducing orifice of the vaginal wall without generating any physical pain or psychological stress. This minimal cross-section, in combination with adequate length, provides for flexibility of the distal section in a plane perpendicular to the axis of introduction, so that the distal section is capable of deforming for adapting to the anatomical shape of the vaginal cavity. This is an important advantage especially for the population of young users, not yet familiar with the operation, and which do not perfectly control the angle of introduction to follow so as to allow for easy insertion of the tampon into the vaginal cavity.

Of course, such a dilator can be designed to adapt to any type of tampon, regardless of the dimension thereof. It will thus be possible to introduce tampons of different sizes and diameters with the same dilator.

As the tampon no longer makes contact with the vaginal wall during intromission thereof, it is no longer necessary to always offer tampons having a profiled distal end so as to minimize the unpleasant feeling perceived by the user. Indeed, due to a dilator according to the invention, the feeling perceived by the user will be the same regardless of the profile of the tampon's distal end. It will thus be possible, or even desirable, to use tampons having a more flared distal end, and thus being less compressed as with conventional tampons, so as to allow for faster and more efficient absorption of the natural effluents making contact with said distal end.

It should be noted that the user will be able either to make the tampon slide into the dilator by means of a finger, or to use a plunger.

Preferably, the cross-section of the internal passage is smaller than 5 mm when the distal section is in the initial resting state. The feeling of penetration perceived by the user is minimized thereby.

According to a preferred embodiment of the invention, the proximal section comprises a distal end area shaped so as to define a passage the cross-section of which is substantially decreasing in the direction of the distal section. In use, the tampon is thus compressed against the substantially tapered distal end area under the effect of the stresses applied to the tampon by the user's finger or by a plunger, which eventually allows for decompression/flaring of the distal end of the tampon, and thus allows for faster and more efficient absorption of the natural effluents making contact with said distal end.

Advantageously, the proximal section has, in the distal end area, a side wall comprising at least one area of internal rugosity. Such an area of rugosity allows to assist in fraying of the distal end of the tampon, in view of enhancing the absorption capacity thereof by an increase of the contact surface.

Also advantageously, the side wall is made as a series of internal shoulders, each having a substantially circular edge forming a n area of rugosity.

Preferably, the distal section comprises a plurality of longitudinal slots delimiting at least two longitudinal tabs designed to have radial spreading elasticity adapted to the cross-section of the tampon. Thus, even if the tampon can make contact with the vaginal wall while it is sliding in the distal section at the longitudinal slots, this contact will be extremely “minimal” and cannot be described as “prolonged”, so that it is totally unlikely to cause any kind of discomfort, irritation, or lesion. Such longitudinal tabs can thus have a very narrow opening area in the initial resting state of the distal section, so as to make the intromission of the dilator much less unpleasant, and move away from each other under the effect of the tampon sliding in the distal section in view of releasing it into the vaginal cavity.

Advantageously, the dilator comprises four longitudinal slots uniformly distributed on the periphery of the distal section.

According to a characteristic embodiment of the invention, the distal end area has a radial spreading capacity greater than that of the longitudinal tabs, e.g. by providing a thinner material thickness in the distal end area than at the longitudinal tabs. When the tampon is sliding, the distal end area can be deployed, and under the action of the area of muscular narrowing of the vaginal wall, the longitudinal tabs can then spread apart uniformly in a radial direction while staying parallel to each other, so that the opening area of the distal end area and that of the longitudinal tabs are substantially the same.

According to another alternative embodiment of the invention, the distal section is subdivided into two elements each having a substantially semicircular base from which a longitudinal tab is projecting. Advantageously, each longitudinal tab is substantially concave. Preferably, both elements are connected to each other at the bases thereof via two diametrically opposed connecting axes. The longitudinal tabs of both elements can thus spread apart when the tampon comes through. Also preferably, each of the two connecting axes is slidingly mounted inside a slot arranged in at least one of the two bases. Thereby, the longitudinal tabs will spread apart uniformly in a radial direction and consequently remain parallel to each other when the tampon is sliding in the distal section.

The present invention also relates to an assembly comprising:
a dilator according to the invention having an elongated proximal section in which a tampon is housed,
a tampon pushing member designed in such a way that it can move from a retracted position, in which it is substantially disposed along the dilator, to an activating position, in which it is disposed as an extension of the dilator.

According to a preferred embodiment of the invention, the pushing member comprises a plunger hinged around a free end element introduced into the proximal end area of the proximal section.

According to a preferred alternative embodiment of the invention, the plunger and the end element are made so that they can be driven by rotation with respect to each other.

Advantageously, the end element is divided into a first block and a second end block, so that, on the one hand, the plunger and the first block can be driven by rotation with respect to each other, and on the other hand, the first block and the second end block can be driven by rotation with respect to each other.

Also advantageously, the second end block comprises at least two contact surfaces projecting outwards.

According to a characteristic of the invention, the assembly comprises a ring inserted into the proximal end area, and in which a housing for the end element of the pushing member is arranged.

Preferably, the housing of the ring has, on the one hand, an open proximal introducing face substantially adapted to the cross-section of the pushing member, and on the other hand, a closed distal end face.

Also preferably, the ring comprises at least one longitudinal channel.

Advantageously, the proximal section comprises an annular gripping member near the distal end area thereof, and this gripping member is provided with a notch to allow for the plunger to be folded back along the dilator.

The implementation of the invention will be better understood by means of the detailed description given below with reference to the appended drawing, wherein:

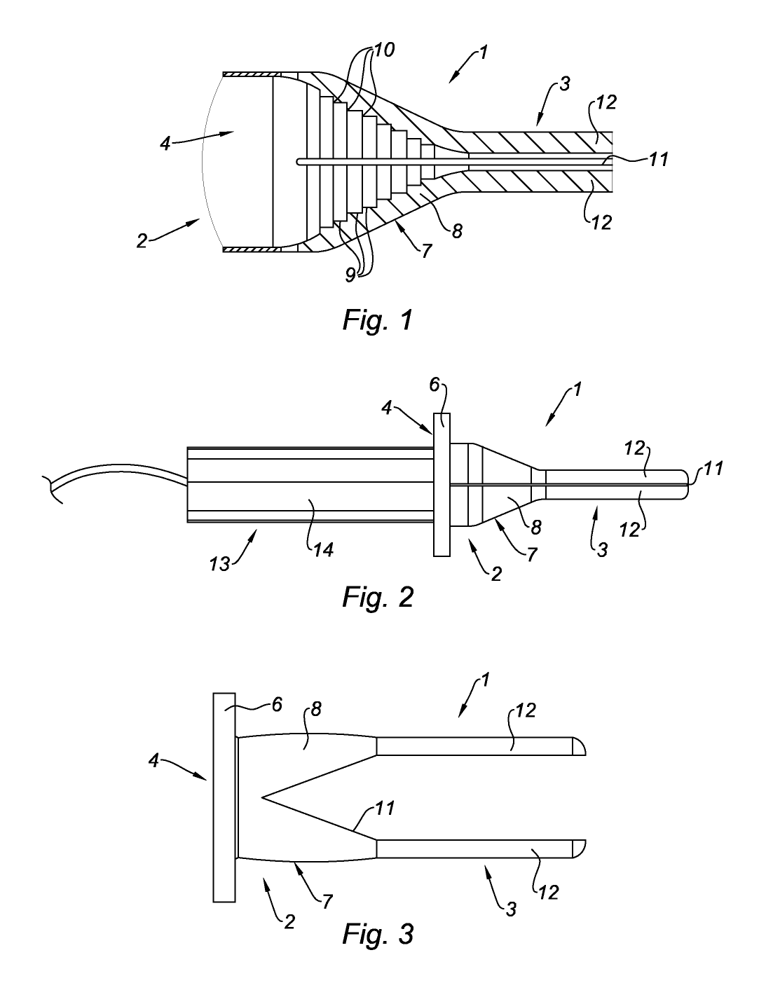

FIG. 1 is a partially enlarged longitudinal sectional view of a dilator according to the invention;

FIG. 2 is a side view of the dilator represented in FIG. 1 with a tampon;

FIG. 3 is a side view of the dilator in the deployed position;

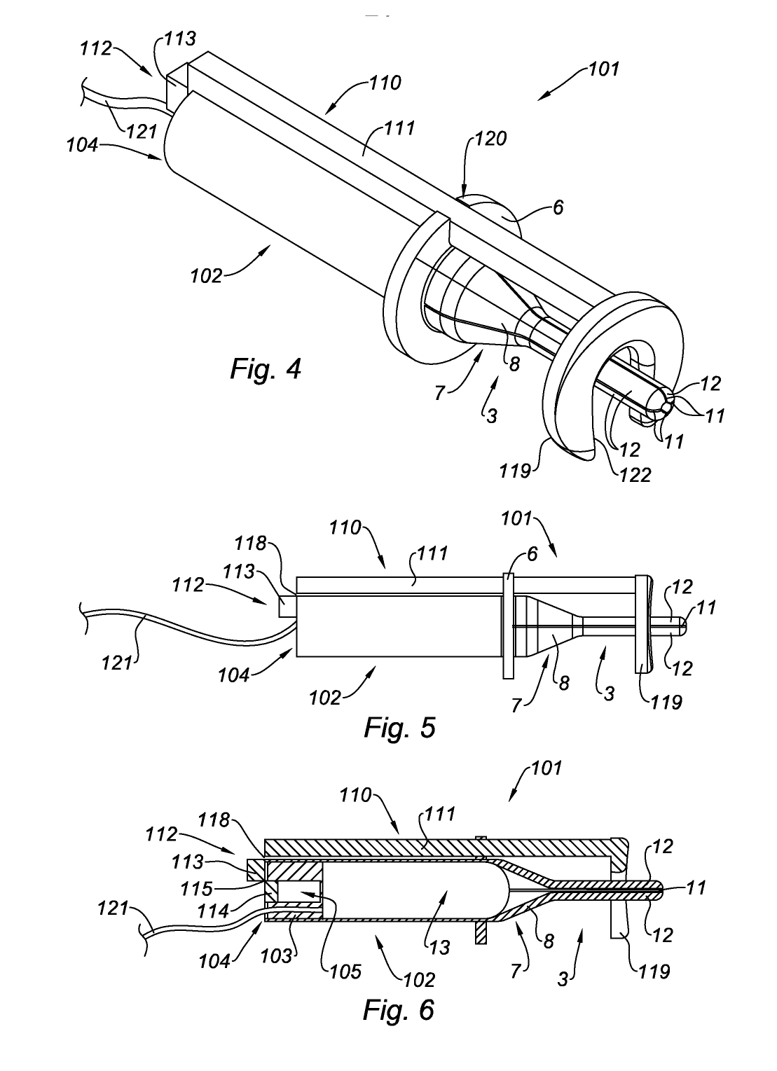

FIG. 4 is a perspective view of the assembly according to the preferred embodiment of the invention, ready for use;

FIG. 5 is a side view of the assembly represented in FIG. 4;

FIG. 6 is a longitudinal sectional view of the assembly represented in FIG. 5;

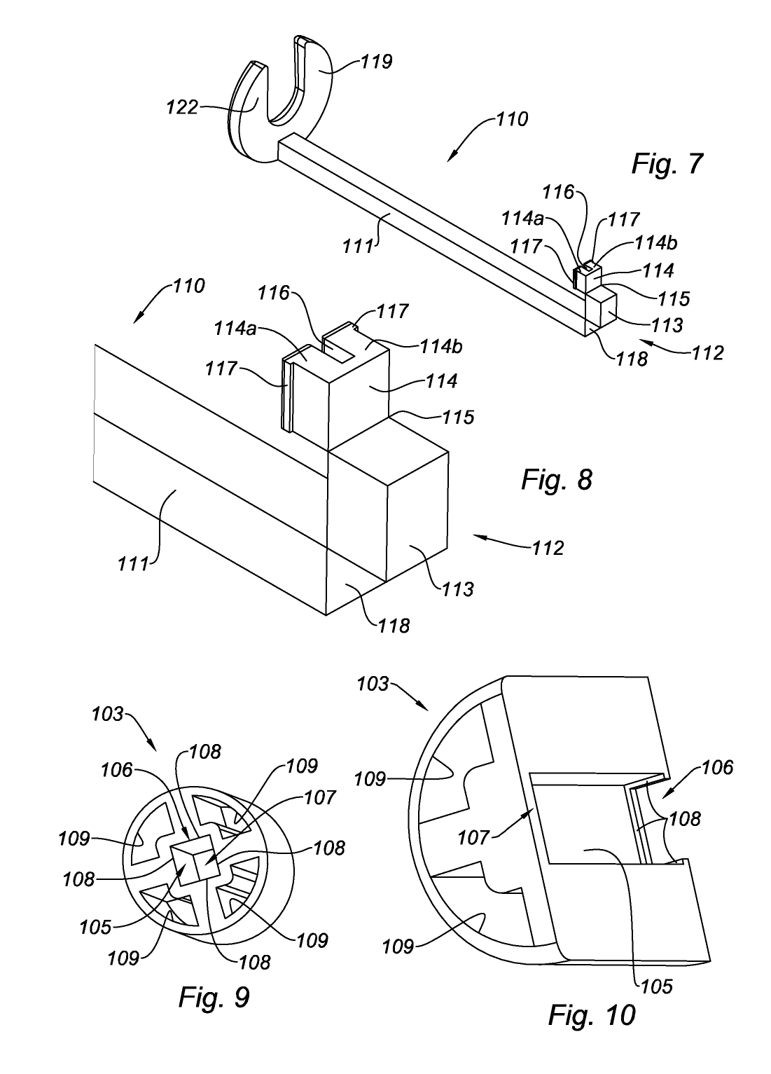

FIG. 7 is a perspective enlarged view of the pushing member of the assembly represented in FIG. 4;

FIG. 8 is a detail view of the end of the pushing member represented in FIG. 7;

FIG. 9 is a perspective view of the ring inserted in the assembly represented in FIG. 4;

FIG. 10 is a sectional enlarged view along line X-X of the ring represented in FIG. 9;

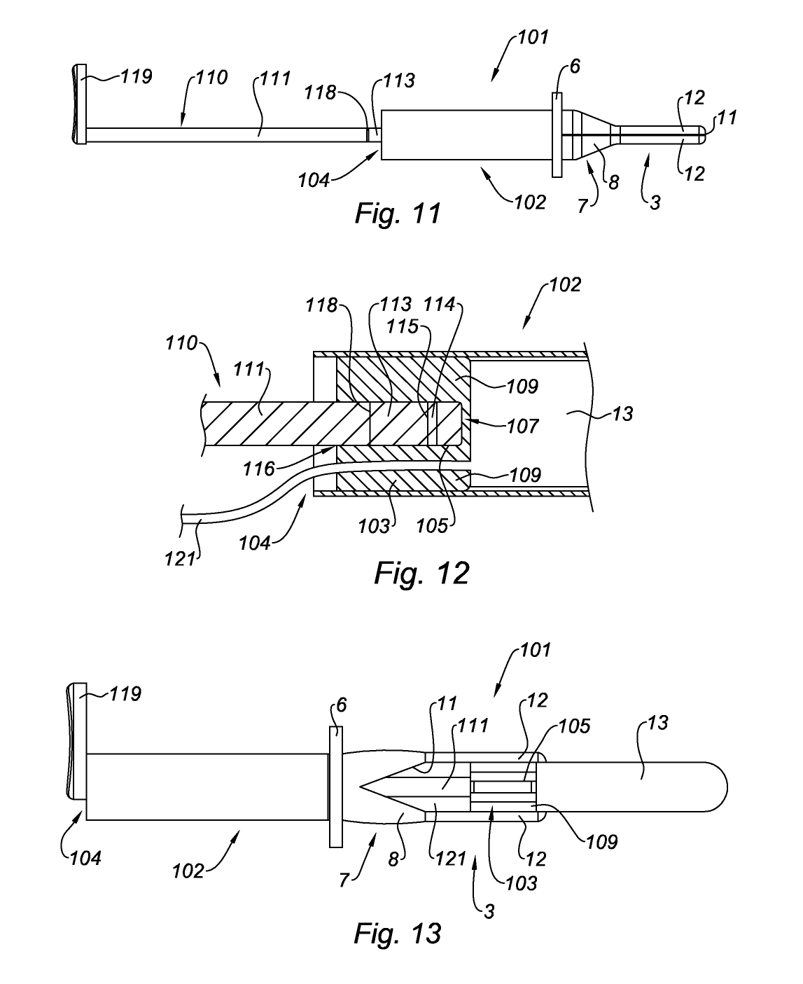

FIG. 11 is a side view of the assembly represented in FIG. 4 once the pushing member has been armed;

FIG. 12 is a longitudinal sectional detail view of the assembly represented in FIG. 11;

FIG. 13 is a side view of the assembly represented in FIG. 4 at the end of the operation;

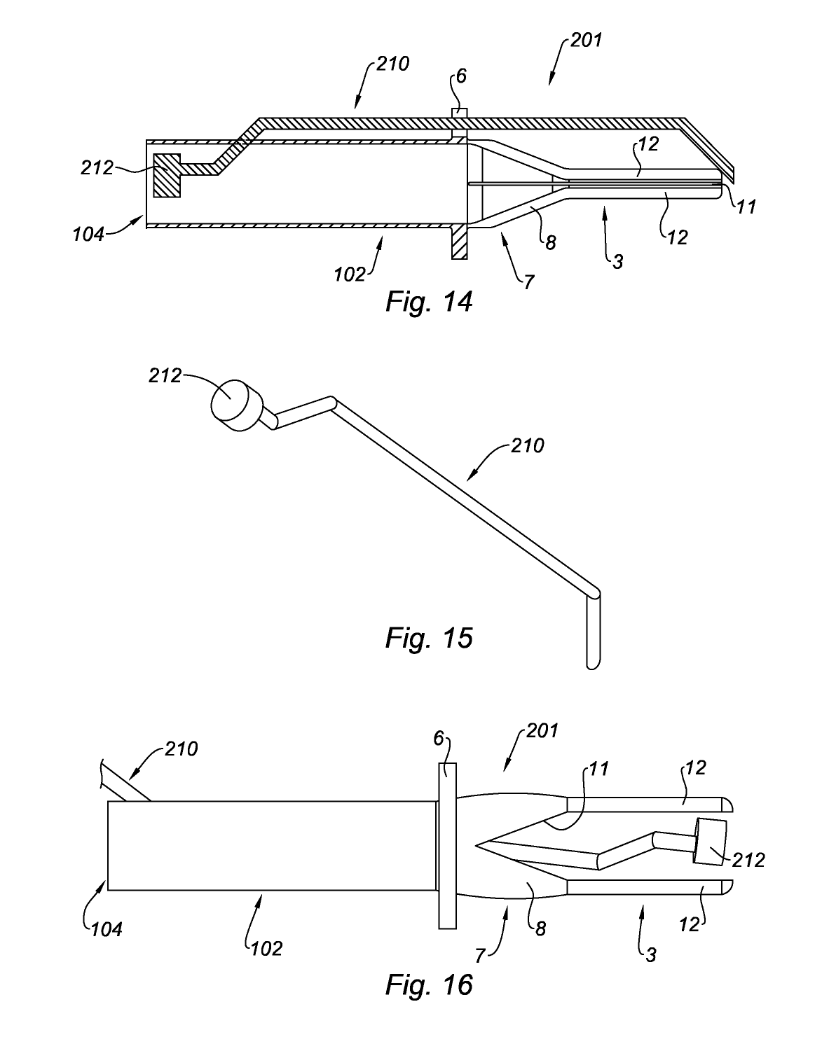

FIG. 14 is a side view of another assembly according to the invention, ready for use;

FIG. 15 is a perspective enlarged view of the pushing member of the assembly represented in FIG. 14;

FIG. 16 is a side view of the assembly represented in FIG. 14 at the end of the operation;

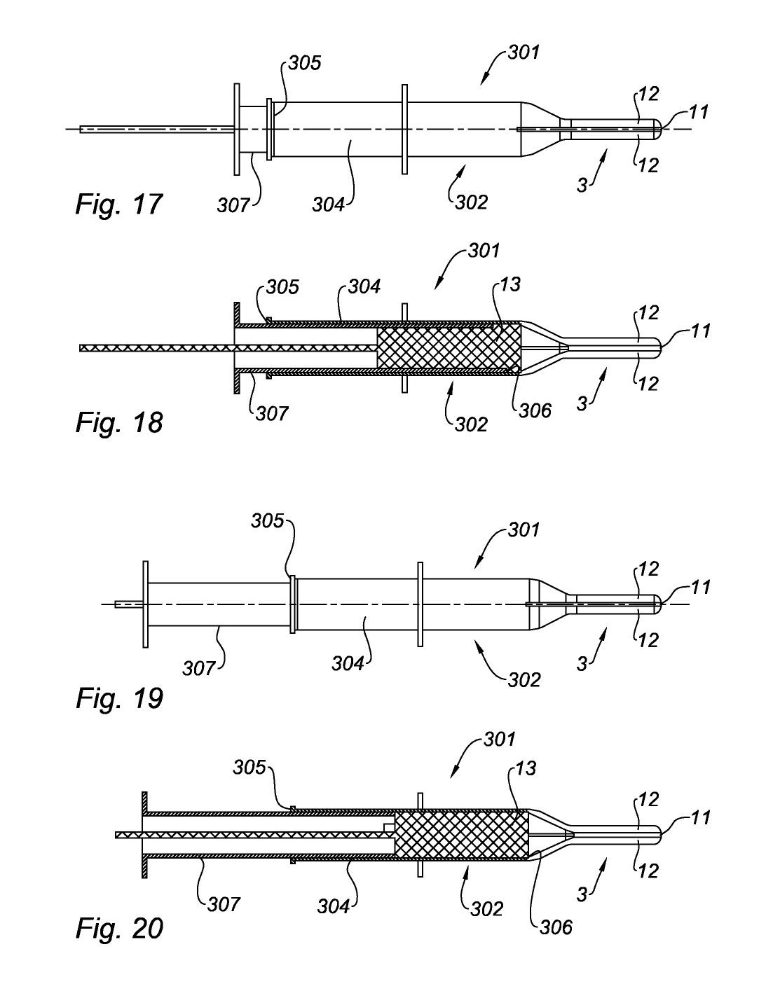

FIGS. 17 and 18 respectively are a side view and a longitudinal sectional view of another assembly according to the invention, ready for use;

FIGS. 19 and 20 respectively are a side view and a longitudinal sectional view of the assembly represented in FIGS. 17 and 18 after the pushing member has been armed;

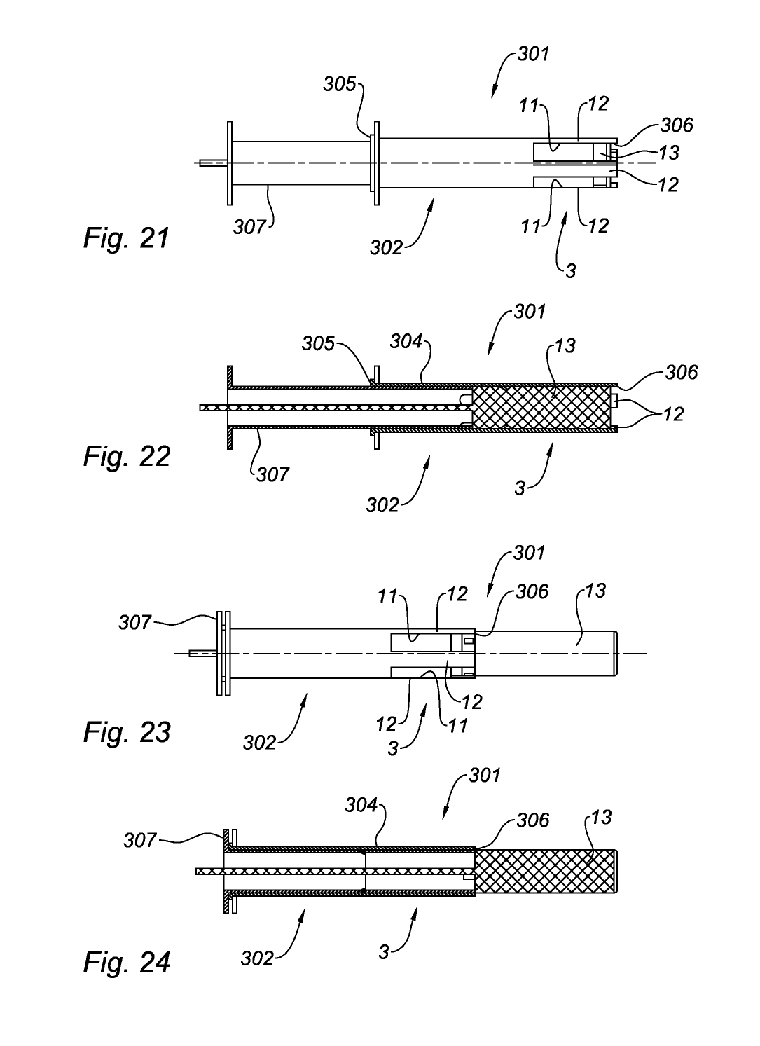

FIGS. 21 and 22 respectively are a side view and a longitudinal sectional view of the assembly represented in FIGS. 17 and 18 when the tampon is being extracted;

FIGS. 23 and 24 respectively are a side view and a longitudinal sectional view of the assembly represented in FIGS. 17 and 18 at the end of the operation;

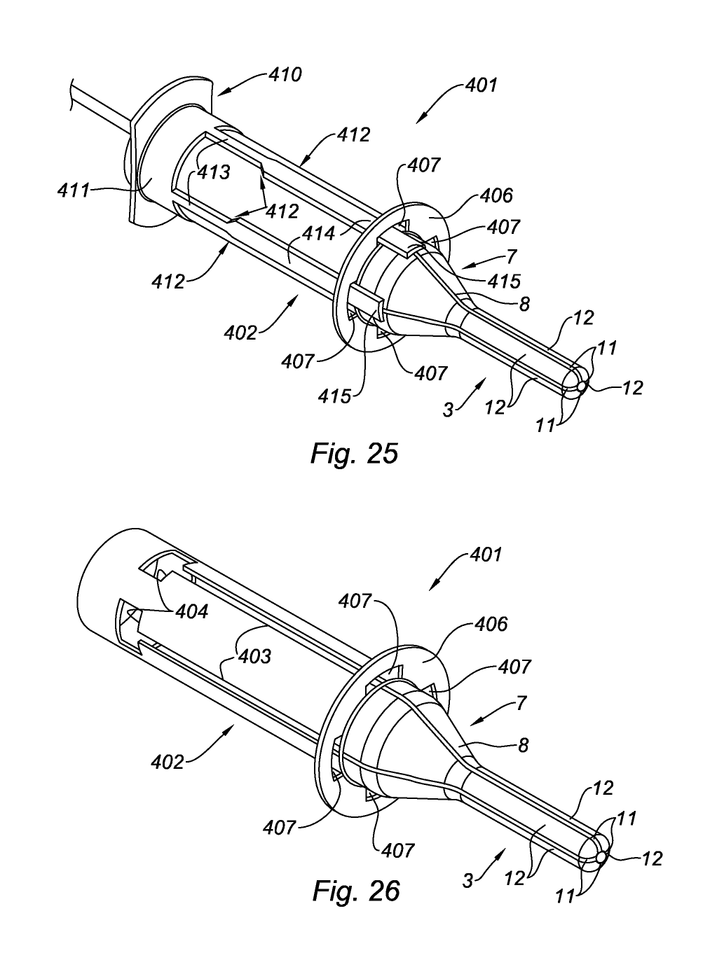

FIG. 25 is a perspective view of another assembly according to the invention;

FIG. 26 is a perspective view of the dilator of the assembly represented in FIG. 25;

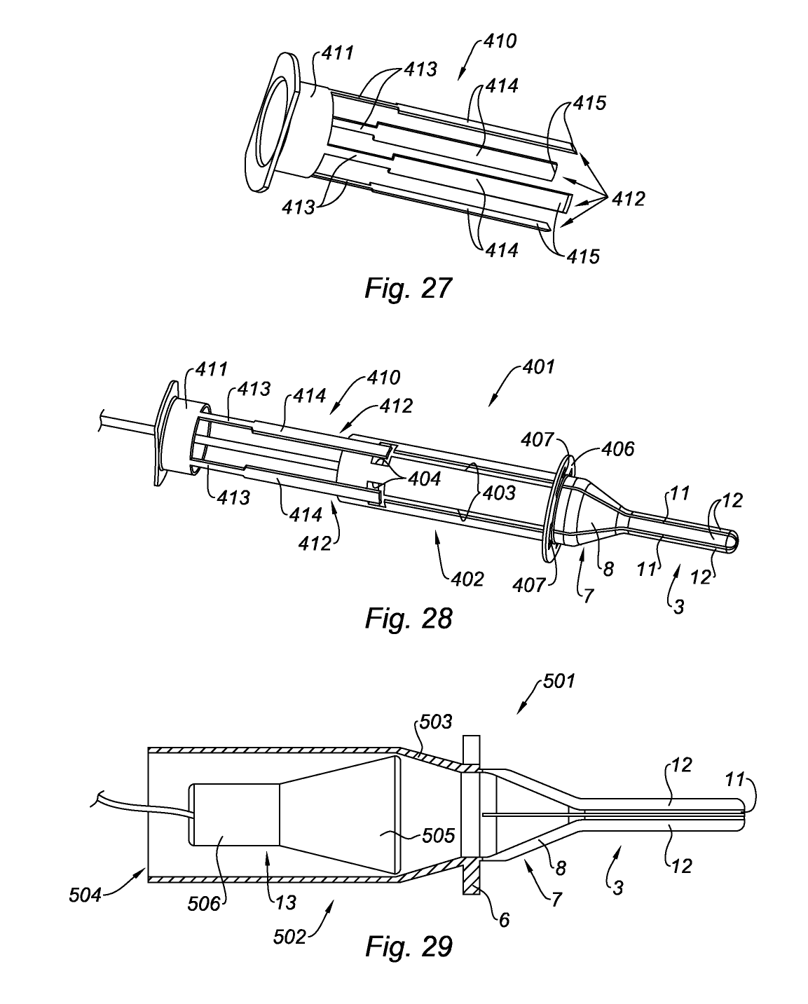

FIG. 27 is a perspective view of the pushing member of the assembly represented in FIG. 25;

FIG. 28 is a perspective view of the assembly represented in FIG. 25 after the pushing member has been armed;

FIG. 29 is a schematic longitudinal sectional view of another assembly according to the invention;

FIGS. 30 to 35 are a side view and a perspective view of three specific kinds of tampons, which can be used with the assembly represented in FIG. 29;

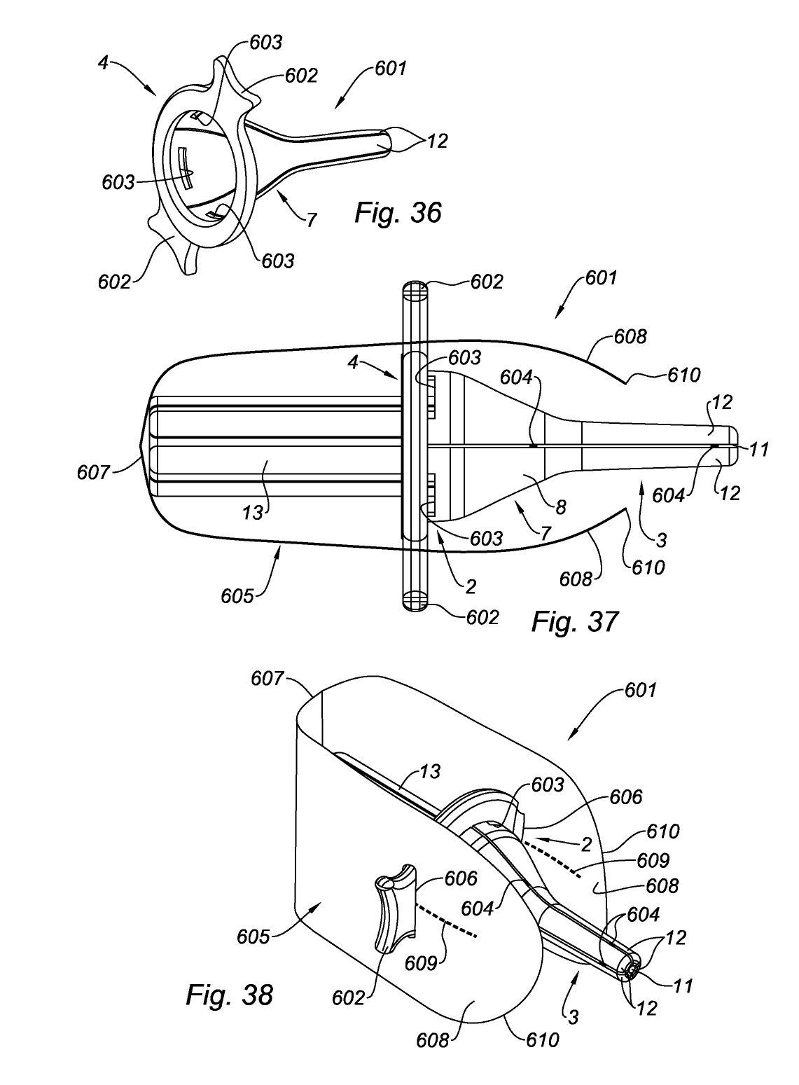

FIG. 36 is a perspective view of another dilator according to the invention;

FIG. 37 is an enlarged side view of an assembly comprising the dilator represented in FIG. 36 fitted with a cover element;

FIG. 38 is a perspective view of the assembly represented in FIG. 37;

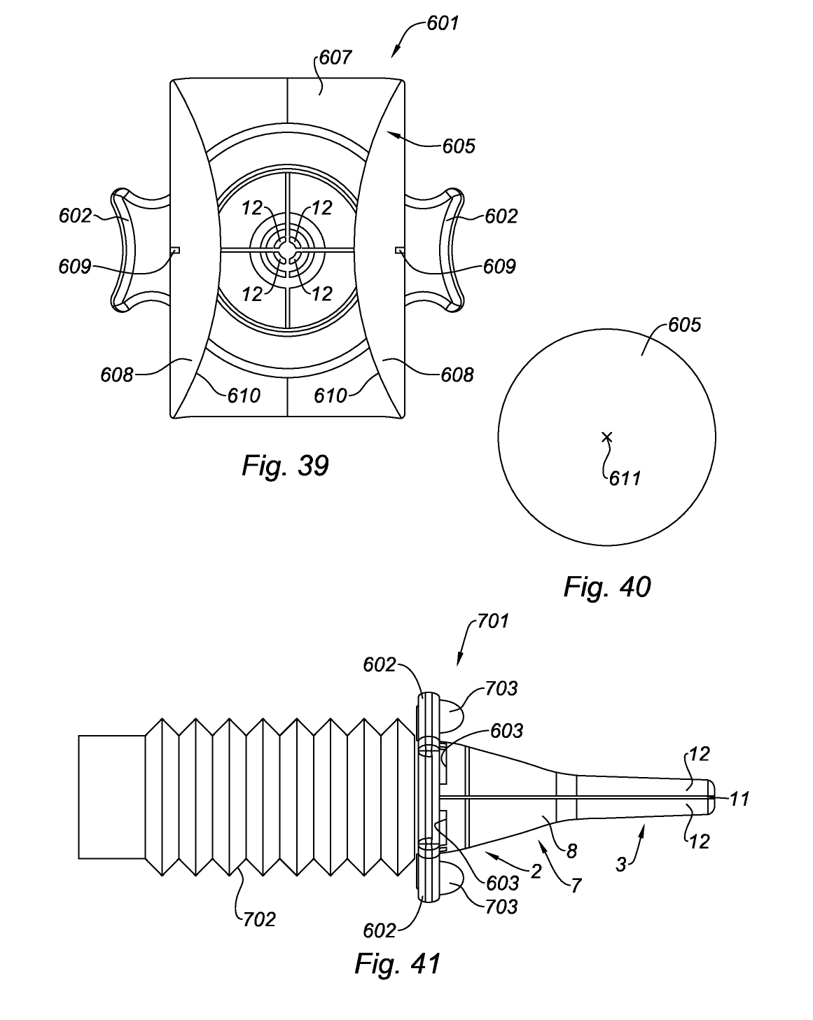

FIG. 39 is a front view of the assembly represented in FIG. 37;

FIG. 40 is a top view of another sample cover element which may be fitted to a dilator represented in FIG. 36;

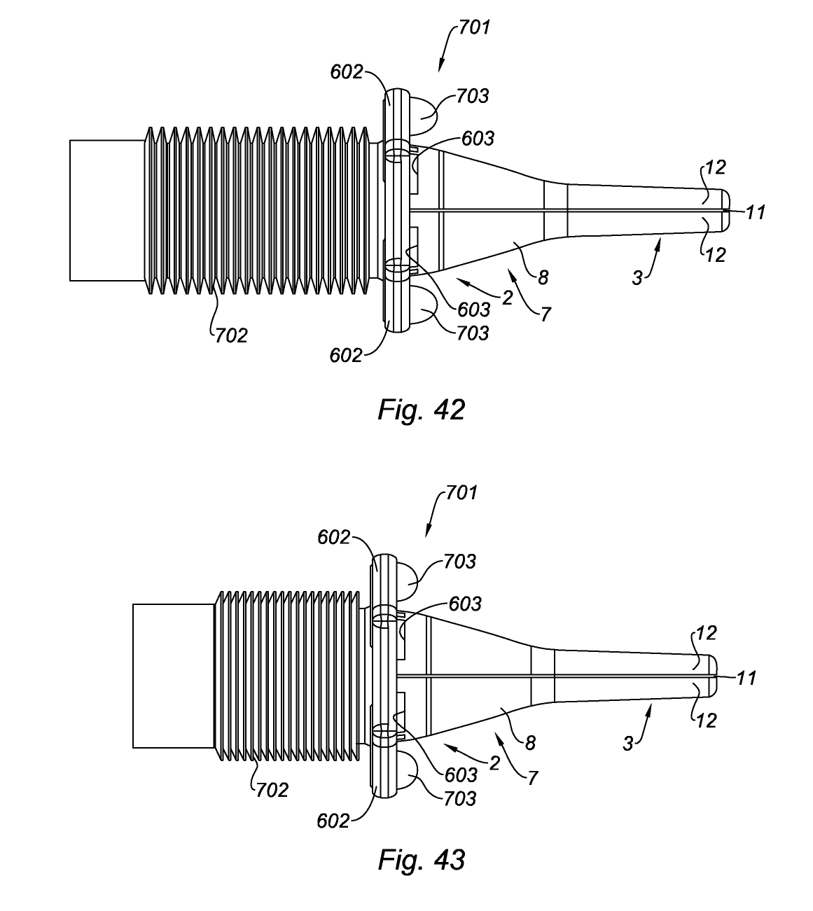

FIGS. 41 to 43 are enlarged side views of another dilator according to the invention, respectively before, during, and at the end of the operation.

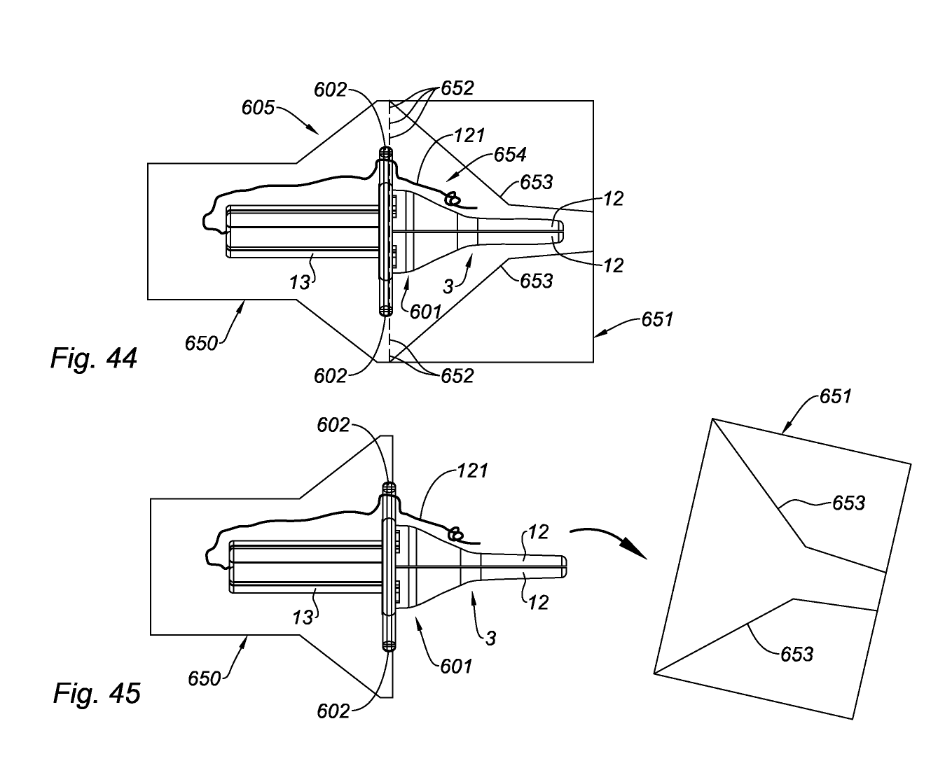

FIGS. 44 and 45 are schematic longitudinal sectional views of another assembly comprising the dilator represented in FIG. 36 fitted with another cover element, before and after use;

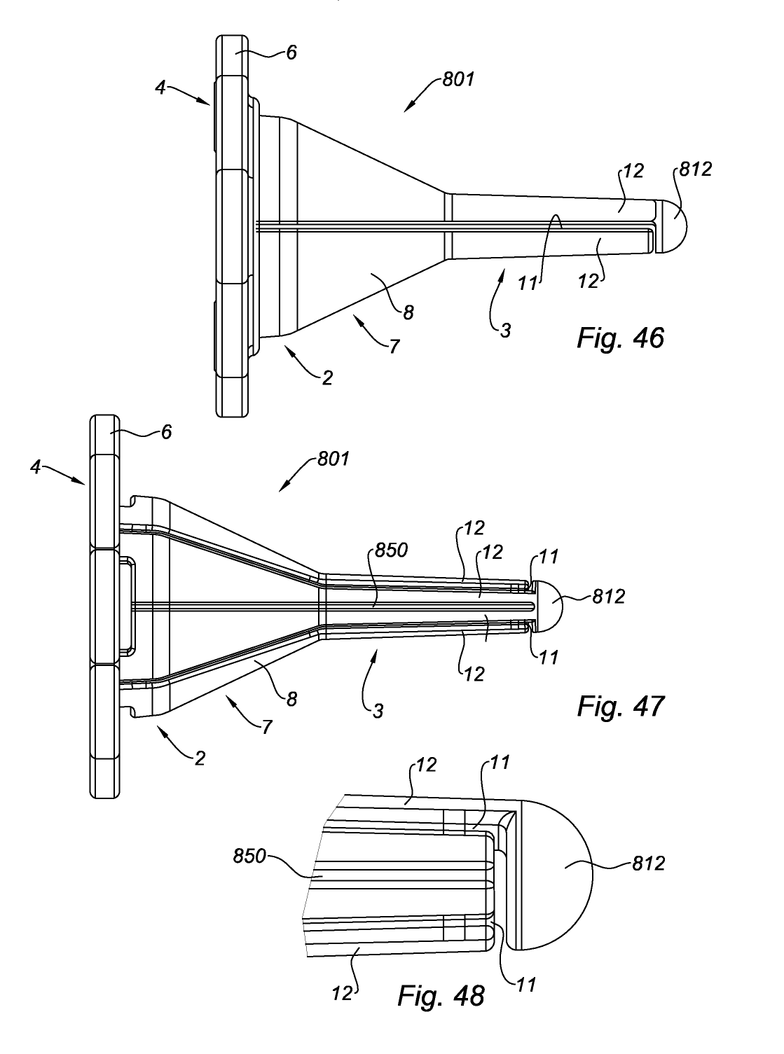

FIG. 46 is an enlarged side view of another dilator according to the invention;

FIG. 47 is a view at another angle of the dilator represented in FIG. 46, fitted with longitudinal grooves;

FIG. 48 is a detail view of the end of the distal section of the dilator represented in FIG. 47;

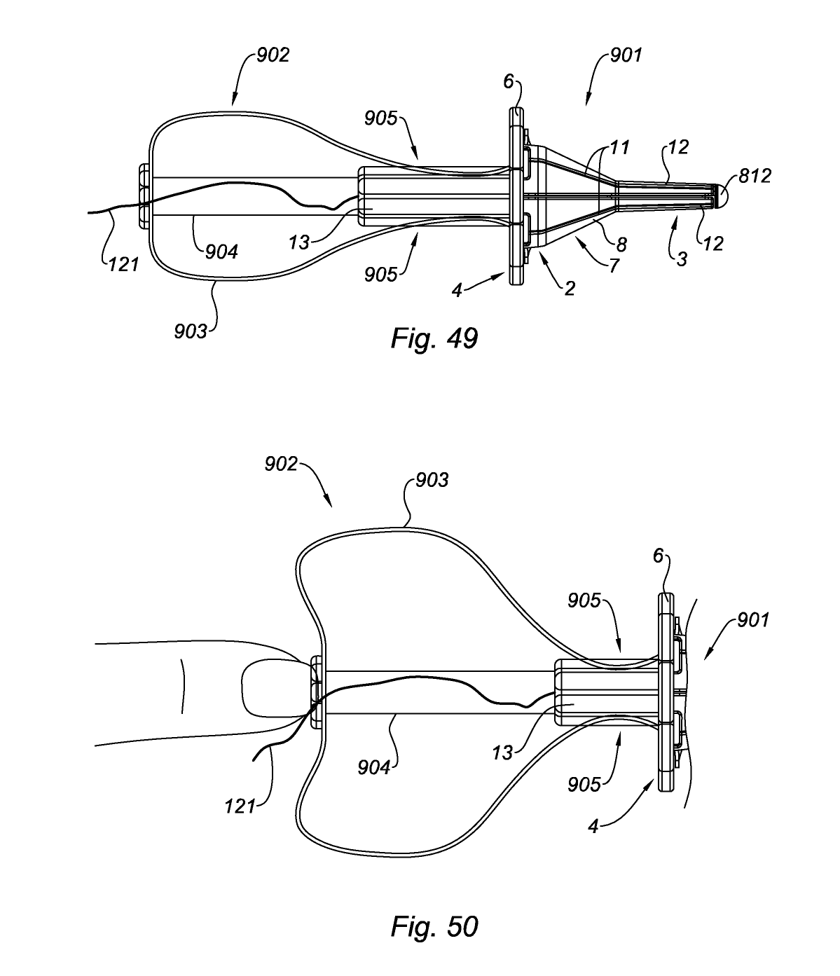

FIGS. 49 to 52 are perspective and side views, some of them partial, of another dilator according to the invention fitted with guiding means, before and after operation;

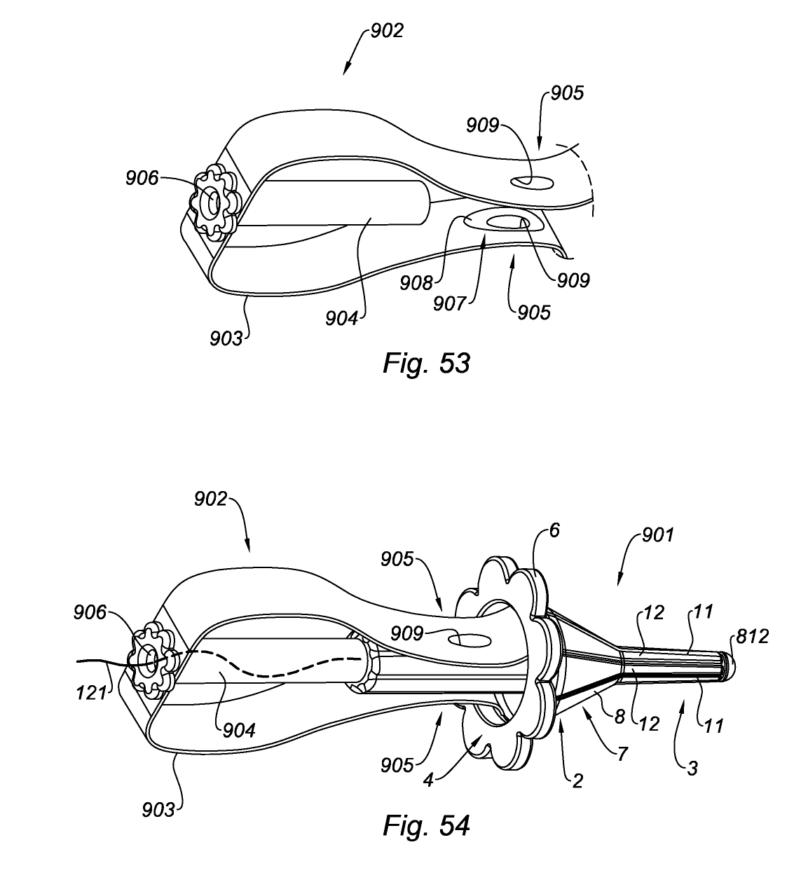

FIGS. 53 and 54 are perspective views of an improvement of the guiding means represented in FIGS. 49 to 52.

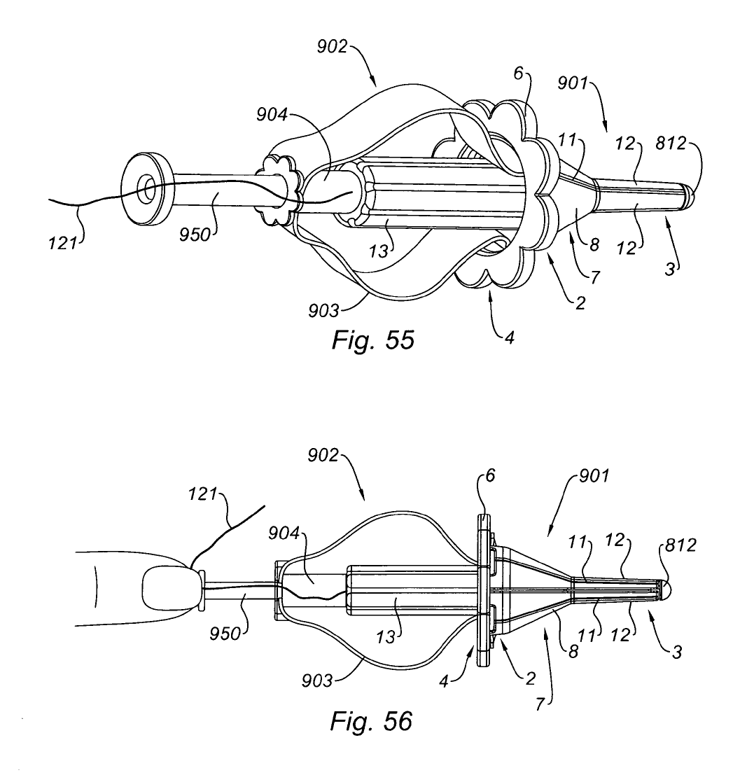

FIG. 55 is a perspective view of a dilator depicted in FIG. 19 fitted with another guiding means;

FIGS. 56 to 60 are side views, some of them partial, of the dilator depicted in FIG. 55, in various stage of deployment.

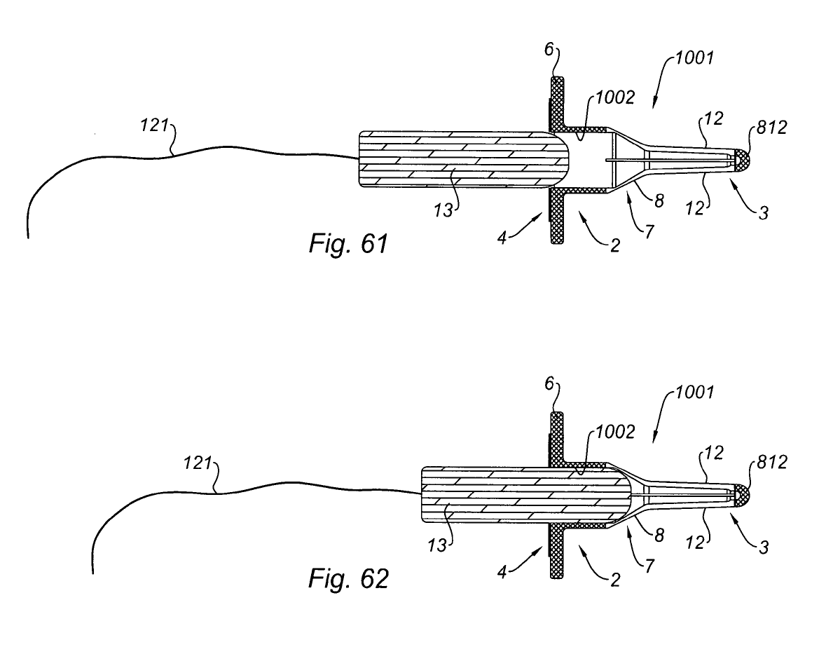

FIGS. 61 and 62 are longitudinal sectional views of another assembly according to the disclosure.

In the following detailed description of the figures defined above, the same elements or elements fulfilling identical functions may keep the same reference symbols in order to facilitate the understanding of the invention.

A dilator 1 according to the present invention, such as represented in FIGS. 1 to 3, comprises an integral body made of biodegradable plastic divided into a proximal section 2 and a distal section 3 respectively having a length on the order of 2 cm and 3 cm.

More precisely, the proximal section 2 is provided with a proximal end area 4 comprising an annular gripping element 6 (not represented in FIG. 1).

The various components of the dilator 1 discussed above, and as described herein, may independently or collectively be formed from a variety of materials, including, for example, plastic, including biodegradable plastic; metal; paper; cardboard; ceramic; glass; and mixtures thereof, unless otherwise specified.

The annular gripping element 6 comprises an inner periphery, an outer periphery, a first major surface, and a second major surface. The inner periphery defines opening, which may be of any size and shape suitable for introducing a tampon into the dilator 1. For example, the shape of the opening may be substantially round, such as the shape of a circle or an oval; may have a polygonal shape; or may be any irregular shape comprising curved and/or straight lines.

The opening defined by the inner periphery of the annular gripping element 6 may have an average diameter, for example, in the range of from about 0.5 to about 10 cm, such as from about 0.5 to about 6 cm, or from about 3 to about 10 cm, or from about 1 to about 3 cm, or from about 1.5 to about 2.5 cm. In some embodiments, the average diameter of the opening is about 1.1 cm, 1.2 cm, 1.3 cm, 1.4 cm, 1.5 cm, 1.6 cm, 1.7 cm, 1.8 cm, 1.9 cm, 2.0 cm, 2.1 cm, 2.2 cm, 2.3 cm, 2.4 cm, 2.5 cm, 2.6 cm, 2.7 cm, 2.8 cm, 2.9 cm, or 3.0 cm.

The opening may have an area, for example, in the range of from about 0.20 to about 80 cm², such as from about 0.20 to about 30 cm², or from about 25 to about 80 cm², or from about 0.80 to about 7 cm², or from about 1.75 to about 5 cm², or from about 2.25 to about 4.5 cm².

The first major surface and second major surface the annular gripping element 6 may be planar or substantially planar in a direction that is perpendicular or substantially perpendicular to the longitudinal direction of interior passageway. Alternatively, the first and second major surfaces and may be angled in a direction either towards or away from the distal end of dilator 1, and/or may not be planar.

The annular gripping element 6 may, for example, have an average thickness from the first major surface to the second major surface in a range of from about 1 to about 10 mm, such as from about 1 to about 6 mm, or from about 4 to about 10 mm, or from about 2 to about 4 mm. The thickness may be uniform, substantially uniform, or variable throughout the annular gripping element 6.

The proximal section 2 comprises a distal end area 7 shaped so as to define a passage, the cross-section of which is substantially decreasing in the direction of the distal section 3. In the distal end area 7, the proximal section 2 has a side wall 8 made as a series of internal shoulders 9 each having a substantially circular edge 10 forming an area of rugosity.

The distal section 3 and the distal end area 7 comprise four longitudinal slots 11 uniformly distributed over the periphery of said dilator 1, thus delimiting four longitudinal tabs 12 designed to have a radial spreading elasticity adapted to the cross-section of a hygienic tampon 13. More precisely, the distal end area 7 has a radial spreading capacity greater than that of the longitudinal tabs 12 due to the fact that the material thickness chosen for making the distal end area 7 is thinner than that chosen for making the longitudinal tabs 12.

In operation, the user takes hold of a tampon 13 and introduces it into the proximal section 2 by the proximal end area 4 until the distal end of the tampon 13 comes into abutment against the side wall 8 of the distal end area 7.

The distal section 3 is introduced into the vagina so as to emerge directly into the vaginal cavity where the tampon 13 is to be delivered. If required, the longitudinal tabs 12 can be made to deform by flexion in a plane perpendicular to the axis of introduction so as to adapt to the anatomical shape of the vaginal cavity. This will further reduce the feeling of discomfort that might be perceived by the user.

By exerting sufficient pressure on the proximal end of the tampon 13, e.g. with the thumb, while performing counter-pressure by positioning e.g. the forefinger and the middle finger around the gripping element 6, sliding of the tampon 13 will then successively cause the distal end area 7 and the longitudinal tabs 12 to spread apart.

Considering their configuration in the resting state, first of all, the edges 10 allow for the distal end of the tampon 13 to be frayed and/or flared under the effect of the pressure exerted on the latter, which eventually enables faster and more efficient absorption of the natural effluents making contact with said distal end of the tampon 13. On the contrary, once the tampon 13 has completely started its stroke and thereby caused the complete deployment of the distal end area 7, the opening area of the latter is substantially circular (as apparent from FIG. 3) and the edges 10 no longer offer any resistance to the sliding of the tampon 13. The side surface 14 of the latter is thus preserved from any untimely deterioration.

As represented in FIG. 3, the longitudinal tabs 12 remain parallel to each other when the tampon 13, not represented for clarity's sake, enters into the distal section 3, due to the area of muscular narrowing (not shown) of the vaginal wall which encloses said longitudinal tabs 12 and thus counters spreading thereof.

Another sample dilator 601 according to the invention, as represented in FIGS. 36 to 39, is different from the dilator 1 described before in that it comprises:
a gripping element arranged in the proximal end area 4 in the form of two diametrically opposed lugs 602 oriented perpendicularly to the axis of the dilator 601,
four lumens 603 arranged in the proximal end area 4 and uniformly distributed over the periphery,
junction points 604 for enabling calibrated opening of the longitudinal tabs 12.

More precisely, the four lumens 603 allow to decrease the efforts to be made to cause radial spreading of the longitudinal tabs 12, and thus allow for better control of the opening of the longitudinal tabs 12.

The junction points 604 connect the longitudinal tabs 12 together before operation. For this purpose, and as schematically represented in FIG. 37, each longitudinal tab 12 is initially attached to each of the two adjacent longitudinal tabs 12 thereof in two points, a first one located in the distal end area 7, and a second one near the distal end thereof. Preferably, the junction points 604 are made integrally from the same material as the longitudinal tabs 12.

Due to these junction points 604, the radial opening of the four longitudinal tabs 12 is calibrated. In operation, the user thus starts by introducing a tampon 13 into the dilator 601, then takes hold of the latter by positioning e.g. her forefinger and her middle finger in abutment against the lugs 602, and by exerting pressure on the proximal end of the tampon 13 by means of her thumb so as to make it slide. When sufficient stresses are applied to the tampon 13, the distal end thereof successively causes rupturing of the junction points 604 located in the distal end area 7, then rupturing of the junction points 604 located near the distal ends of the longitudinal tabs. It should be understood that it may be of advantage to reinforce the resistance of the junction points 604 arranged in the distal end area 7 in order to allow for real propulsion of the tampon 13 in the direction of the vaginal cavity once these junction points 604 will have given in.

Furthermore, the dilator 601 is fitted with a cover element 605. More precisely, the latter is e.g. made from a material which may be described either as woven or non-woven, plastic or non-plastic, elastic or non-elastic, impermeable or not-impermeable, soluble or not-soluble, etc. A large variety to choose from is thus offered for making this cover element 605.

In FIGS. 37 to 39, this cover element is chosen as a web 605, which is substantially rectangular with rounded corners, having two slots 606 for fastening said web 605 to the dilator 601.

The tampon 13 is first of all inserted into the dilator 601, as can be seen in FIGS. 37 and 38. The web 605 is then attached to the dilator 601 by inserting each of the two lugs 602 into the slot 606 corresponding thereto. The web 605 is thus tensioned and allows for the tampon 13 to be maintained in the position engaging into the proximal section 2.

Another advantage provided by such a web 605 is due to the fact that this web 605 allows to avoid that the user's finger used for pushing the tampon 13 is soiled by the flows from the vaginal cavity. Indeed, the center part 607 of the web 605 will intervene between the finger and the tampon 13 throughout sliding of said tampon 13, so that this finger will never make contact with the vaginal cavity.

In a similar fashion, excess material 608, located between each slot 606 and the end 610 of the web 605 corresponding thereto, is used to protect the tip of the finger positioned in abutment against the corresponding lug 602 from any soiling, by intervening between said finger and the lips of the genital organ.

Also, each of the two excess materials 608 has a longitudinal line of weakness 609 which progressively tears up upon contact with the corresponding lug 602, as the tampon 13 slides into the dilator 601, so that the web 605 can accompany the tampon 13 over any length of the stroke thereof until it is released into the vaginal cavity.

Alternatively, the cover element can be embodied as a disk 605 as represented in FIG. 40. Herein, this disk 605 covers the whole tampon 13 put in place, and thereby is advantageously provided with a cross-shaped center lumen 611 for letting pass the withdrawal cord of the tampon 13.

Of course, alternatively, the cover element 605 can be embodied as a piece of material having for instance a substantially square, triangular, or even trapezoidal shape.

Furthermore, fastening the cover element 605 to the two lugs 602 can alternatively be obtained e.g. as the result of a welding operation.

Finally, the cover element 605 can advantageously be coated with the following kind of products: e.g. disinfectant for fingers, cicatrizer, lubricant; or else with a product providing pH control.

Another sample embodiment is represented in FIG. 44. More precisely, the cover element 605 is made to totally cover the dilator 601 and the tampon 13 fitted with the withdrawal cord 121 thereof. Thereby, the cover element 605 also fulfils the function of a complete packaging bag for the assembly formed by the dilator 601 and the associated tampon 13.

This cover element 605 has a proximal part 650 covering the tampon 13 engaged in the dilator 601, and which is fastened to the dilator 601 at the lugs 602.

However, this fastening is made so that an opening is at least liberated for the passage of the free end of the withdrawal cord 121 in the direction of the distal end of the dilator 601.

The withdrawal cord 121 can thus pass an orifice arranged e.g. in or near one of the lugs 602.

Alternatively, the withdrawal cord 121 can simply be slipped into a space arranged between the dilator 601 and the proximal part 650. In this case, fastening the proximal part 650 to the dilator 601 has to be done in a discontinued fashion.

This fastening is advantageously made by welding, but obviously, any other kind of fastening compatible with the subject technical field can be used instead of welding.

The proximal part 650 is extended by a distal part 651 having a substantially rectangular shape covering the dilator 601. This distal part 651 is integral with the proximal part 650, and the areas of weakness 652 are arranged so that the distal part 651 can be detached from the proximal part 650 in a predetermined fashion. In this instance, the areas of weakness 652 are located substantially inside the plane of the lugs 602.

However, it is obvious that such areas of weakness 652 could be arranged elsewhere, and especially around the distal section 3 of the dilator 601. Such areas of weakness 652 can in this case be arranged advantageously, but not necessarily, in a transverse plane to the longitudinal tabs 12.

The distal part 651 can comprise partially oblique welding areas 653 which allow to arrange a funnel-shaped housing 654 for the dilator 601. Thereby, the longitudinal tabs 12 of the dilator 601 are engaged in the frayed part of the housing 654, which prohibits any untimely displacement of the dilator 601 fitted with the associated tampon 13 inside the cover element 605.

When a user wants to perform the intromission of a tampon 13, she takes hold of the cover element 605 used as a packaging bag.

Next, using one hand, she will exert traction on the distal part 651, while maintaining with her other hand either the free end of the proximal part 650, or the lugs 602 through the proximal part 650. The distal part 651 is thus made to tear up at the areas of weakness 652, as represented in FIG. 45, and can afterwards be disposed of (as indicated by the arrow) in a waste bin disposed nearby, or even tossed into the toilet bowl if the material used for making the cover element allows therefor. The proximal part 650 remains integral with the dilator 601.

Finally, the user can really perform the insertion of the tampon 13 into her vaginal cavity by pushing the proximal end of the tampon 13 via the proximal part 650, still being protected from any soiling due to said proximal part 650. As the tampon 13 slides along, the free end of the withdrawal cord 121 will be secured to stay outside of the dilator 601. Alternatively, the proximal part 650 can be fitted with a pushing member. The latter can e.g. be embodied as a substantially cylindrical body having, on the one hand, a first end attached to said proximal part 650, and on the other hand, a second end making contact with the tampon 13. When the user exerts pressure on the proximal part 650, the pushing member will then make the tampon 13 slide.

An assembly according to the preferred embodiment of the invention, as represented in FIGS. 4 to 13, comprises a dilator 101 having an integral body made of biodegradable plastic divided into a proximal section 102 and a distal section 3 substantially identical to that of the dilator 1 described before. The proximal section 102 is different from the proximal section 2 described before mainly due to the fact that, on the one hand, it has a length on the order of 7 cm allowing it to completely house the tampon 13, and on the other hand, the gripping member 6 comprises a notch 120.

After the tampon 13 has been inserted into the proximal section 102, a ring 103 is slipped into the proximal section 102 by a proximal end area 104 thereof. This ring 103 has a longitudinal center housing 105 having a square cross-section comprising an open proximal introducing face 106 and a closed distal end face 107. The proximal introducing face 106 also has a square cross-section, and is fitted with ribs 108 so as to make the opening area of said proximal introducing face 106 slightly smaller than that of the housing 105. The ring 103 further comprises four longitudinal channels 109 disposed around the center housing 105.

Prior to inserting the ring 103 into the proximal section 102, an integral pushing member 110 having a square cross-section, made of biodegradable plastic and comprising a plunger 111 hinged around a free end element 112, is inserted into the ring 103.

This end element 112 is divided into a first block 113 and a second end block 114 disposed one after the other, and attached to each other via a material thread 115, as can be seen in particular in FIG. 8. The first block 113 and the second end block 114 can thus be driven by rotation with respect to each other.

The second end block 114 is divided into two symmetrical segments 114a, 114b by means of a slot 116, and each segment 114a, 114b comprises a contact surface 117 oriented outwards. The second end block 114 is shaped so that the cross-section thereof, due to the two contact surfaces 117, is slightly greater than that of the proximal introducing face 106 of the ring 103. The plunger 111 is also connected to the first block 113 via a material thread 118, so that the latter can thus be driven by rotation with respect to each other. At the opposite end thereof, the plunger 111 is fitted with a pushing element 119 comprising an opening 122.

Thus, the second end block 114 is forcibly inserted into the housing 105, with the segments 114a, 114b converging towards each other under the stress, and returning to their initial resting state after the contact surfaces 117 have moved past the ribs 108. The second end block 114 thus ends up being inserted inside the housing 105, unable to leave it, in as far as the ribs 108 act as a stop with regard to the contact surfaces 117. The first block 113 stays outside the proximal section 102, so that the plunger 111 can be folded back into the retracted position along the dilator 101, as represented more particularly in FIG. 4. The plunger 111 finally enters into the notch 120 of the gripping member 6 to be as close as possible to the dilator 101, and thus minimize the global volume of the assembly in the resting position.

Finally, the withdrawal cord 121 of the tampon 13 is aspirated by means of an appropriate tool so that it passes one of the four channels 109 and is thereby accessible from the outside of the assembly.

A user wishing to use the assembly starts by arming the pushing member 110 by deploying the plunger 111. For this purpose, the latter is rotated at an angle of about 180° around the material thread 118, and the first block 113 in turn is also rotated around the material thread 115.

Once the pushing member 110 is completely deployed and the plunger ends up as an extension of the dilator 101, as represented in FIG. 11, the user can exert pressure on the pushing element 119. In doing so, the pushing member 110 will slide until the second end block 114 comes into abutment against the distal end face 107 of the ring 103. At this time, the two material threads 115 and 118 end up inside the housing 105, as represented in FIG. 12, which prohibits any pivoting of the first block 113 with respect to the second end block 114, as well as any pivoting of the plunger 111 with respect to the first block 113. Thereby, the pushing member 110 is blocked in the maximum deployment position thereof by means of the housing 105 of the ring 103.

If a sufficient stress is still exerted on the pushing element 119, the ring will be translated by the pushing member 110 along the proximal section 102, and will in turn force the tampon 13 to slide in the direction of the distal section 3. Extraction of the tampon 13 is then done in an identical manner as already described before.

It should be noted that the ring 103 remains captive of the distal section 3 after the tampon 13 has been released under the effect of the longitudinal tabs 12 which tend to close, as represented in FIG. 13.

It should be noted that the cross-section of the proximal section 102 may alternatively be e.g. rectangular, square, triangular, or else hexagonal. The shape of the ring 103 may also be adapted accordingly.

An alternative of this assembly is represented in FIGS. 14 to 16. Indeed, according to this alternative, the assembly is different due to the fact that, on the one hand, the dilator 201 does not comprise any ring, and on the other hand, the pushing member 210 is rigid, has a substantially bent shape, and has a cylinder-shaped free end element 212. As before, the pushing member 210 is armed by forcing the latter to pivot according to an angle of about 180°. Extraction of the tampon 13 is done by translating the pushing member 210, so that the free end element 212 makes the tampon 13 slide along the dilator 201 until it is extracted from the vaginal cavity.

Another alternative embodiment of an assembly according to the invention is represented in FIGS. 17 to 24. In this sample embodiment, the dilator 301 is different from the dilator 1 only in that the proximal section 302 is more elongated. As for the distal section 3, it remains unchanged.

The assembly also comprises a tube 304 having a proximal end 305 and a distal end 306.

The dilator 301 is shrunk fit onto the distal end 306. A tampon 13 is inserted into the tube 304, then a hollow plunger 307 is introduced into the tube 304 by the proximal end 305 thereof. This plunger 307 is intended to cause the displacement of the tampon 13 along the tube 304 and the dilator 301.

It should be noted that the cross-section of the proximal section 302 may alternatively be e.g. rectangular, square, triangular, or else hexagonal.

Another alternative embodiment of an assembly according to the invention is represented in FIGS. 25 to 28. In this example, the dilator 401 is different from the dilator 1 mainly in that the proximal section 402 is elongated, comprises four identical longitudinal slots 403 evenly distributed over the circumference and each having an enlarged proximal end 404, and has a gripping member 406 fitted with four orifices 407 disposed respectively as an extension of the four slots 403. The distal section 3 remains unchanged.

The assembly also comprises a hollow pushing member 410 divided into a base 411 extended by four longitudinal tabs 412 uniformly distributed over the circumference. Each tab 412 comprises a proximal part 413 having a reduced cross-section and a distal part 414 having an enlarged cross-section, the latter being chosen to be smaller than the cross-section of the proximal end 404, but greater than that of slot 403. This distal part 414 ends in a free end 415 curved inwards.

As represented in FIG. 25, the pushing member 410 is previously positioned around the dilator 401. The tabs 412 have sufficient radial elasticity to really enclose said dilator 401. The free ends 415 each pass the corresponding orifice 407. A tampon (not represented) is inserted into the dilator 401.

A user wishing to use the assembly starts by arming the pushing member 410 by pulling the same. During the translation, the tabs 412 will slide on the slots 403 without being able to go therethrough because of the dimensions of their respective cross-sections. At the end of arming, and as represented in FIG. 28, the free end 415 of the tabs 412 emerges in the enlarged proximal end 404.

When the user exerts pressure on the base 411, the free ends 415 will first of all slide under the slots 403 because the tabs 412 tend to return to their resting position, then slide inside the proximal section 402 over the entire length of the slots 403. During this movement, the tabs 412 cannot leave the slots 403 because of the dimensions of their respective cross-sections.

It should be noted that the cross-section of the proximal section 402 may alternatively be e.g. rectangular, square, triangular, or else hexagonal.

Another sample embodiment of an assembly according to the invention is represented in FIG. 29. In this assembly, the dilator 501 is different from the dilator 101 described before mainly in that the proximal section 502 has an enlarged diameter, and comprises an annular area having a tapered cross-section 503 sandwiched between the proximal end area 504 and the distal end area 7. By doing so, it is possible to use any kind of tampon, and namely a tampon having a large diameter, being less compressed, and as such not adapted to the opening area of the distal end area 7. Indeed, In this case, the annular area having a tapered cross-section 503 will serve for compressing the tampon when it is sliding, so that this tampon inside the distal end area 7 has a diameter adapted to the latter. Illustratively, a tampon having a diameter of 14 mm can be used instead of a conventional tampon having a diameter on the order of 12 mm.

The tampon 13 inserted into the dilator 501 comprises a flared distal end 505 in the shape of a truncated cone, and a proximal end 506 having a conventional cross-section. It should be noted that such a tampon 13 is advantageous in that the flared distal end 505 offers a much greater contact surface than that offered by a standard tampon. This is all the more interesting as it is during the first minute after the insertion of a standard tampon into the vaginal cavity that blood can flow or slide along the tampon because the latter has not yet been overly compressed.

It should be noted that the cross-section of the proximal section 502 can alternatively be e.g. rectangular, square, triangular, or else hexagonal.

In FIGS. 30 to 35 three examples of tampons 13 are represented which can be used with an assembly as represented in FIG. 29.

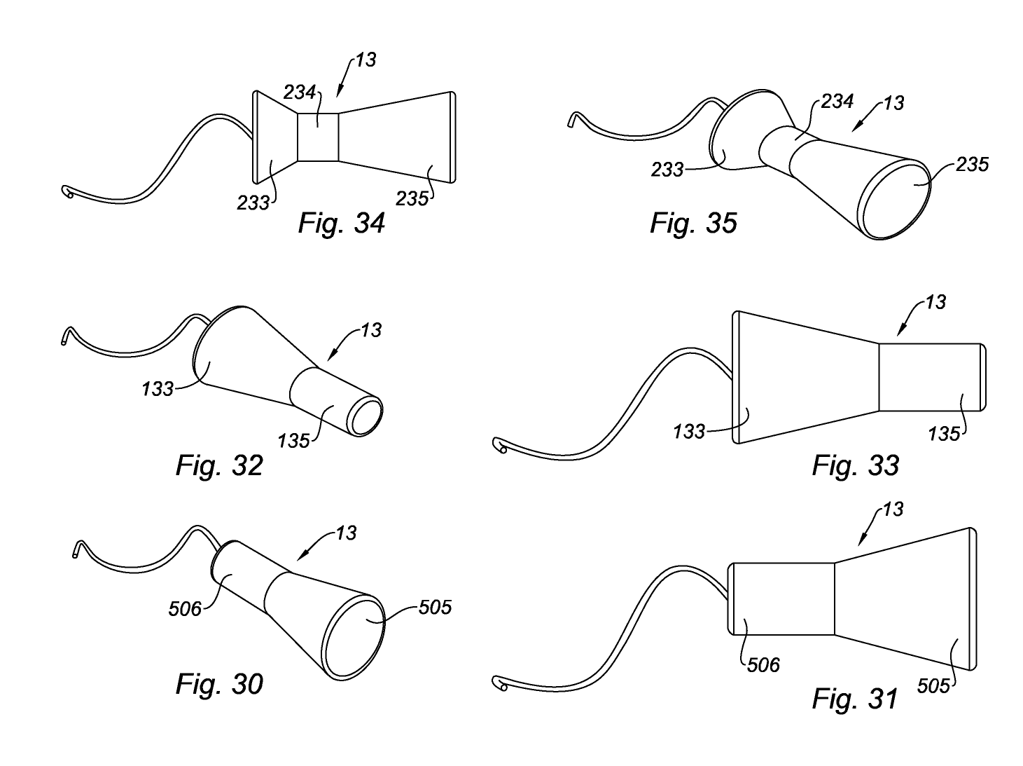

The tampon 13 of FIGS. 30 and 31 corresponds to the one illustrated in FIG. 29.

The tampon 13 represented in FIGS. 32 and 33 is remarkable in that it comprises a flared proximal end 133 in the shape of a truncated cone and a flat distal end 135. Such a tampon is advantageous in that, on the one hand, it allows to decrease the risk of downward leakage, and on the other hand, users having a larger than average vaginal cavity are sure not to lose it.

The tampon 13 represented in FIGS. 34 and 35 has a flared proximal end 233 and a flared distal end 233, in the shape of a truncated cone and disposed on either side of a cylindrical center section 234. Such a configuration allows to combine the advantages discussed before.

Another sample embodiment of a dilator 701 according to the invention is represented in FIGS. 41 to 43.

More precisely, in this embodiment, the proximal section 2 is fitted with a telescopic longitudinal member 702 into which a tampon can be slipped.

When the user wishes to insert a tampon into her vaginal cavity, she takes hold of the dilator 701 and places two fingers 703, e.g. her forefinger and middle finger, in abutment against the two lugs 602, then exerts pressure on the proximal end of the longitudinal member 702 by means of her other finger, e.g. the thumb (not represented). By doing so, the longitudinal member 702, initially deployed, will be compressed until it reaches the position of maximum compression represented in FIG. 43.

In case a pushing member (not represented) is added to cause the displacement of the tampon inside the longitudinal member 702 and the proximal section 2, using a telescopic longitudinal member 702 allows for the overall length of the pushing member to be reduced by a distance equal to the difference of length between the deployed position and the position of maximum compression of the longitudinal member 702. Thereby, the assembly is still easy to use.

It should be noted that for the sake of simplicity of the drawing, the longitudinal tabs 12 of the dilator 701 are represented only in the resting position. Obviously, these longitudinal tabs 12 would have to be in the opening position when the longitudinal member 702 is being compressed and the tampon has started to slide along the distal section 3.

Another sample embodiment of a dilator 801 according to the invention is represented in FIGS. 46 to 48. This dilator 801 differs from the dilator 601 namely in that one of the four longitudinal tabs 12 has a free end extended by a semispherical body 812 oriented so as to cover, in the resting position, the free ends of the three other longitudinal tabs 12. These other three free ends are not attached to the body 812.

Due to the convex profile thereof, this body 812 also allows to:
play the part of a centering/guiding member during insertion of the distal section 3 into the vagina. This option offered by the dilator 801 is much appreciated, especially by young users who are not yet familiar with the operation of intromission of a tampon;
guarantee for the user, by covering the free ends of the three other longitudinal tabs 12, that none of these other free ends will be capable of causing any injury or irritation, or even as much as pinching the vaginal wall during the intromission of the tampon.

As represented more particularly in FIGS. 47 and 48, this dilator 801 may also comprise at least one longitudinal groove 850 hollowed out inside each of the four longitudinal tabs 12.

The object aimed at is to further facilitate the insertion of the distal section 3 into the vaginal cavity. Indeed, it has been found to be preferable for this purpose to reduce the surface of the dilator making contact with the vaginal wall, namely by arranging shallow cavities in the distal section 3.

The grooves 850 are thus an example of this kind of cavities allowing to decrease the resistance to sliding, but it should be understood all the same that these cavities could alternatively be made in any other shape.

Furthermore, making such grooves 850 is not limited to the embodiment of the dilator 801, but could more generally be envisaged in all of the embodiments allowing therefor.

It should be noted that for the sake of simplicity of the drawing, the longitudinal tabs 12 of the dilator 801, as well as the longitudinal tabs 12 of most dilators described in the present application, are represented only in the resting position. Obviously, these longitudinal members 12 would be in the opening position when tampon 13 has started to slide along the distal section 3.

Another dilator 901 according to the invention, as represented in FIGS. 49 to 53, substantially corresponds to the dilator 801, fitted with spring-loaded guiding means 902 attached to the proximal section 2.

More precisely, these guiding means 902 are made in the shape of a loop spring blade 903, and the two ends of which are integral with the proximal end area 4. Advantageously, the dilator 901 and the spring blade 903 will be made integrally during the same molding operation.

This spring blade 903 has a pushing member 904, embodied in the shape of a cylindrical body the length of which substantially corresponds to the length of the dilator 901.

Before operation, and as represented in FIG. 49, a tampon 13 is introduced into the proximal end area 4 of the dilator 901. The spring blade 903 is bent so as to have two bending areas 905 allowing to enclose the tampon 13 and center the same in the proximal end area 4. This support is further improved due to the other end of the pushing member 904 which comes to rest on the end of the tampon 13 so as to “set” the same in the storage position. The string 121 of the tampon 13 is slipped into the pushing member 904, and the free end thereof emerges from the orifice 906 of the pushing member 904.

At the beginning of operation, and as represented in FIG. 50 (the dilator 901 is represented only partially), the user exerts pressure on the spring blade 903 at the orifice 906, e.g. by means of her forefinger. The spring blade 903 is compressed, causing the translation of the tampon 13 via the pushing member 904.

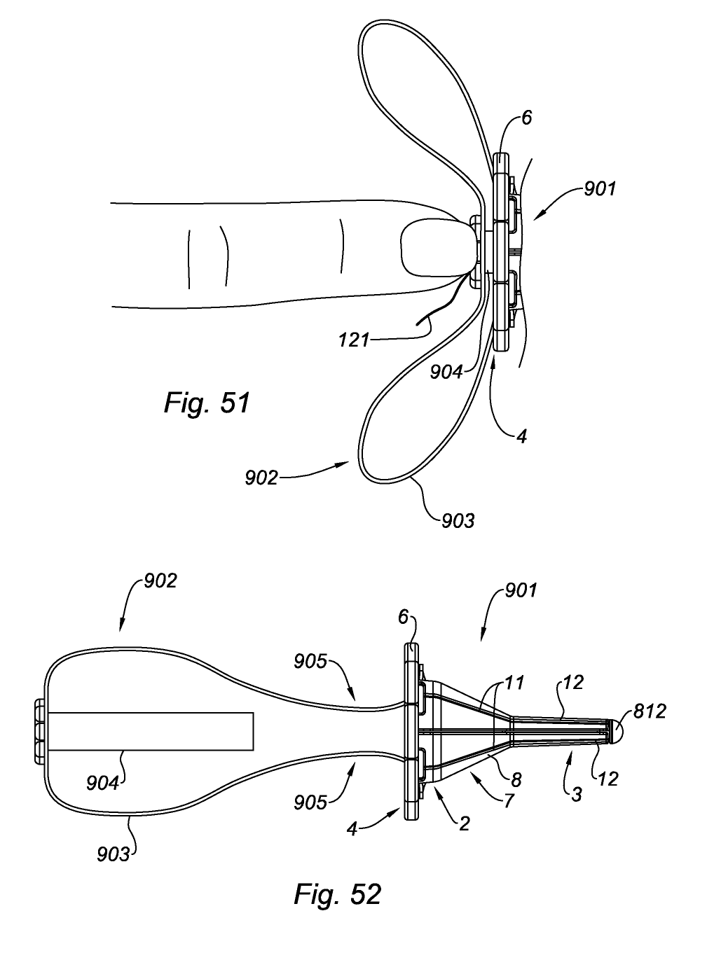

At the end of the pressure, and as represented in FIG. 51 (the dilator 901 is represented only partially), the spring blade 903 substantially comes into abutment against the proximal end area 4. The length of the pushing member 904 being substantially equal to that of the dilator 901, with the result that the tampon 13 is then expelled from the dilator 901 so as to be adequately delivered into the vaginal cavity.

A major advantage of this version is due to the fact that the user is protected from any soiling as her fingers are not at all made to penetrate inside the dilator 901 or inside the vaginal cavity.

The user can then release the pressure she exerted on the spring blade 903, this which produces the effect of letting the latter return by elasticity the initial resting position thereof, as represented in FIG. 52. The pushing member 904 is driven by the spring blade 903, and this backward movement of the pushing member 904 releases the string 121 which so far was housed inside the pushing member 904. At this time, the dilator 901 in turn has also returned to its initial resting position, in which the diameter of the distal section 3 is minimal.

The user can finally remove the dilator 901. This operation of withdrawal is hardly perceived by the user because of the minimal diameter of the distal section 3, which is a very significant advantage. The string 121 is completely released from the dilator 901 during backward movement of the latter.

Advantageously, and as represented in FIGS. 53 and 54, the spring blade 903 comprises a cutout 907 arranged in the inner surface of each of the two bending areas 905 so as to further improve the support of the tampon 13. More precisely, each cutout 907 has a concave border 908 surrounding a center through orifice 909. Thereby, each cutout 907 allows for part of the side surface of the tampon 13 to be perfectly enclosed. Certain fibers composing the tampon 13 can also slightly stand out from the center orifice 909, so as to further improve “the anchoring” of the tampon 13 in the storage position thereof.

Another substantial advantage of such a version is due to the fact that the overall mass of the assembly is minimal due to the small amount of material used for making the different components.

Obviously, the guiding means 902 can be embodied in a variety of shapes. As for a spring blade 903, it might for instance have only a single spring-loaded tab. This spring blade 903 could alternatively be embodied in the shape of a loop comprising several branches evenly distributed around the circumference of the tampon 13 in order to further reinforce support of the latter.

Alternatively, the guiding means 902 can be embodied in the shape of distinct mechanical parts made integral with the dilator 901, e.g. by snap-fitting, ratchet motion, gluing, welding, etc.

Referring to FIGS. 55 to 60, in some embodiments, the guiding means 902 may further comprise a plunger 950. In these embodiments, pushing member 904 and/or loop spring blade 903 is configured to define a passageway whereby at least a portion of plunger 950 is slidably positioned within the passageway.

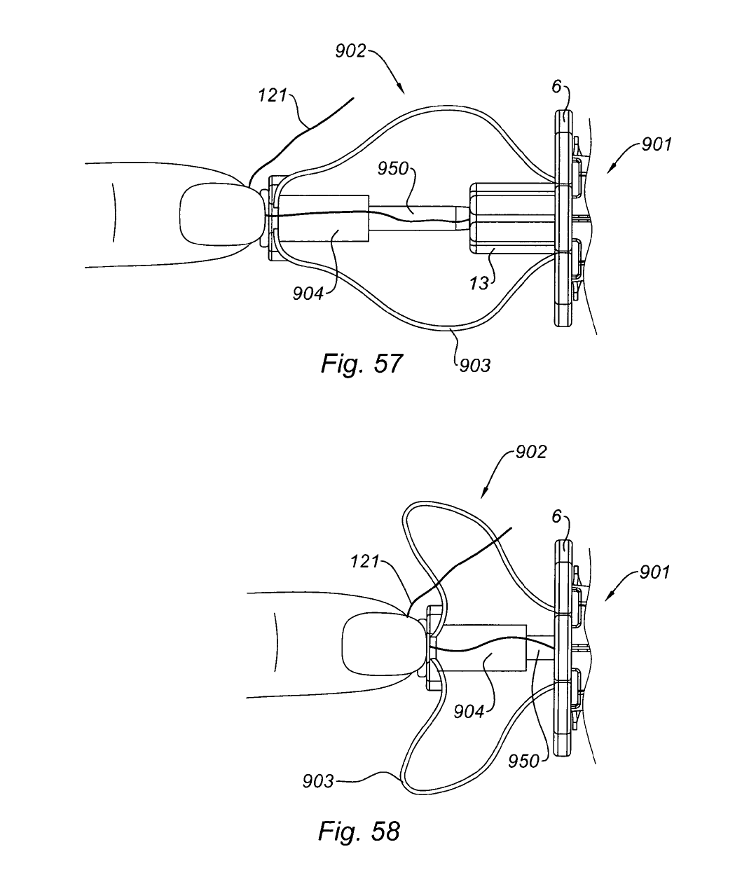

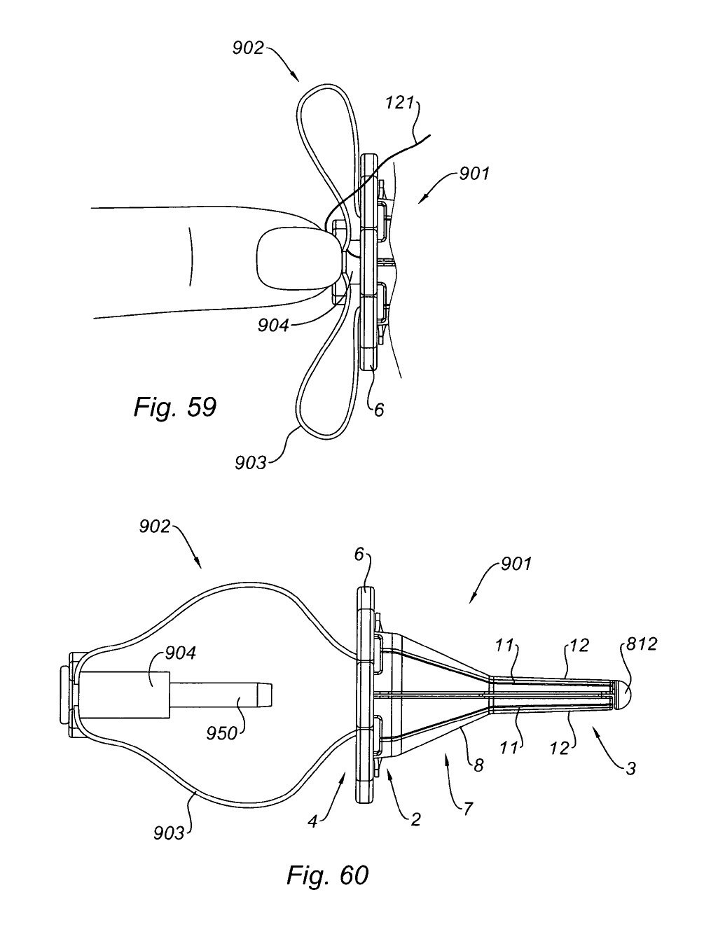

The plunger 950 may be configured so that the distal end of the plunger 950 may be pushed beyond the distal end of pushing member 904 and/or loop spring blade 903 to a distance so that the length of the pushing member 904 plus the length of the plunger 950 is sufficiently long to completely expel a tampon 13 from the dilator 901 into the vaginal cavity. Before operation, and as represented in FIG. 56, the distal end of a tampon 13 is introduced into the proximal end of the dilator 901. The distal end of the plunger 950 is placed in contact with the proximal end of tampon 13. Using, for example, a thumb or a forefinger, the user may push on the proximal end of plunger 950 to exert a force on the proximal end of tampon 13 to begin pushing the tampon 13 through the dilator 901. As this happens, plunger 950 slidably moves through pushing member 904 and/or loop spring blade 903 so that the proximal end of the plunger 950 moves towards the proximal end of pushing member 904 while the distal end of plunger 950 moves away from the distal end of pushing member 904. During this stage, the distal end of loop spring blade 903 may not be deformed towards annular gripping element 6. The plunger 950 continues its free movement relative to the pushing member 904 until a portion of plunger 950 engages with a portion of member 904/903, as shown in FIG. 57, so that the application of sufficient force on the proximal end of plunger 950 causes the plunger 950 and member 904/903 to move together while simultaneously pushing the proximal end of loop spring blade 903 towards annular gripping element 6, as represented in FIG. 58. As depicted in FIG. 59, the guiding means 902 may be configured so that the entire or substantially entire length of the pushing member 904 can enter into the interior passageway of dilator, and the distal end of loop spring blade 903 substantially comes into abutment against the annular gripping element 6. Upon release of the force applied to the loop spring blade 903, the resiliency of loop spring blade 903 urges the loop spring blade 903 to return to its original or substantially original position thus pulling out pushing member 904 and plunger 950 from the interior passageway of the dilator 901, as shown in FIG. 60, so as not to impede the return of longitudinal members 12 of the dilator 901 to their initial resting position as the user withdraws the dilator 901 from the vaginal opening.

In any of the embodiments discussed above, the interior passageway may be provided or fitted with a bushing member (not depicted) that narrows the opening size by which a tampon can be introduced. The bushing may be configured to provide additional support to a tampon introduced into the dilator, or to help guide the tampon more centrally to the interior passageway. The bushing may be permanently secured to the dilator, or be configured to be removable.

Referring to FIGS. 61 and 62, in addition to, or as an alternative to using a bushing member, a dilator 1001 may further comprise an elongated tube section 1002 between the distal end area 7 and the annular gripping element 6 configured to provide additional support to a tampon 13 introduced into the dilator 1001, and/or to help guide the tampon 13 more centrally and more parallel to the longitudinal axis of the interior passageway. Although elongated tube section 1002 as depicted is cylindrical, the cross-sectional shape of the elongated tube section 1002 may alternatively be, for example, rectangular, square, triangular, hexagonal, or any other polygonal shape, or any other shape comprising straight and/or curved lines.

Although the invention has been described in relation to the specific sample embodiments, it is obvious that is in no way limited thereto, and that it comprises all the technical equivalents of the means described as well as the combinations thereof, if they fall into the scope of the invention. Also, it should be noted that a dilator according to the invention could also be used for delivering any kind of body into one of orifices presented by the human body, and namely for delivering suppositories.

1. A dilator comprising a body having at least a proximal section provided with a proximal end area by which can be introduced a ready-to-use hygienic tampon, and a distal section intended to be introduced into the vaginal wall of a user, said distal section being shaped so that:
on the one hand, it can switch from an initial resting state, in which it delimits a longitudinal internal passage, the cross-section of which is smaller than the cross-section of the tampon, to a deployed state, in which the cross-section of the internal passage adapts to the cross-section of the tampon, so as to allow for the longitudinal movement of said tampon,
on the other hand, it is long enough to allow for release of the tampon directly into the vaginal cavity, without said tampon being able to make any prolonged contact during intromission thereof with the area of the muscular narrowing of the vaginal wall.

2. The dilator according to claim 1, wherein the section of the internal passage is smaller than 5 mm when the distal section is in the initial resting state.

3. The dilator according to claim 1, wherein the proximal section comprises a distal end area shaped so as to define a passage, the cross-section of which is substantially decreasing in the direction of the distal section.

4. The dilator according to claim 3, wherein in the distal end area, the proximal section has a side wall comprising at least one internal area of rugosity.

5. The dilator according to claim 4, wherein the side wall is made in the shape of a series of internal shoulders each having a substantially circular edge forming an area of rugosity.

6. The dilator according to claim 1, wherein it comprises a plurality of longitudinal slots delimiting at least two longitudinal tabs designed to have spreading radial elasticity adapted to the cross-section of the tampon.

7. The dilator according to claim 6, wherein it comprises four longitudinal slots evenly distributed over the periphery of said dilator.

8. The dilator according to claim 6, wherein the distal end area has a radial spreading capacity greater than that of the longitudinal tabs.

9. An assembly comprising:
dilator according to claim 1 having an elongated proximal section in which a tampon is housed,
a pushing member of the tampon designed so that it can switch from a retracted position, in which it is substantially disposed along the dilator, to an activating position, in which it is disposed as an extension of the dilator.

10. The assembly according to claim 9, wherein the pushing member comprises a plunger hinged around a free end element introduced into the proximal end area of the proximal section.

11. The assembly according to claim 10, wherein the plunger and the end element are embodied to be able to be driven by rotation with respect to each other.

12. The assembly according to claim 11, wherein the end element is divided into a first block and a second end block, so that, on the one hand, the plunger and the first block can be driven by rotation with respect to each other, and on the other hand, the first block and the second end block can be driven by rotation with respect to each other.

13. The assembly according to claim 12, wherein the second end block comprises at least two contact surfaces projecting outwards.

14. The assembly according to claim 9, wherein it comprises a ring inserted in the proximal end area and in which is arranged a housing for the end element of the pushing member.

15. The assembly according to claim 14, wherein the housing of the ring has, on the one hand, an open proximal introducing face substantially adapted to the cross-section of the pushing member, and on the other hand, a closed distal end face.

16. The assembly according to claim 15, wherein the ring comprises at least one longitudinal channel.

17. The assembly according claim 9, wherein the proximal section comprises an annular gripping member near a distal end area thereof, and that this gripping member is provided with a notch to allow for the plunger to be folded back along the dilator.

18. The assembly according to claim 3, wherein it comprises an annular area having a tapered cross-section section sandwiched between the proximal end area and the distal end area.

19. An assembly comprising a dilator according to claim 1, fitted with spring-loaded guiding means.

20. The assembly according to claim 19, wherein the guiding means comprise a pushing ember of the tampon.

21. The assembly according to claim 19, wherein the guiding means are embodied in the shape of a loop spring blade attached to the dilator.

22. An assembly comprising a dilator according to claim 1, which comprises an elongated tube section configured to provide additional support to a tampon introduced into the dilator, and/or to help guide the tampon more centrally and more parallel to the longitudinal axis of the dilator.
